(19) United States
(12) Patent Application Publication
(10) Pub. No.: US 2020/0077939 A1
(43) Pub. Date: Mar. 12, 2020

(54) AUTONOMOUS MULTISENSORY APPARATUS FOR SCREENING AND THERAPY OF VISUAL, AUDITORY AND COGNITIVE IMPAIRMENT WITH DIAGNOSTIC CAPABILITY AND METHOD THEREOF

(71) Applicant: IVISION TECHNOLOGIES, LLC, Des Plaines, IL (US)

(72) Inventors: Stuart Paul Richer, Highland Park, IL (US); Robert Endo, Northbrook, IL (US); Douglas K. Oshana, Lincolnwood, IL (US)

(73) Assignee: IVISION TECHNOLOGIES, LLC, Des Plaines, IL (US)

(21) Appl. No.: 16/563,501

(22) Filed: Sep. 6, 2019

Related U.S. Application Data
(60) Provisional application No. 62/728,044, filed on Sep. 6, 2018.

(51) Int. Cl. A61B 5/00; A61B 5/024; A61B 5/087; A61B 5/0205; A61M 21/02; A61B 5/12; A61B 3/107

(52) U.S. Cl. CPC A61B 5/4088; A61B 5/6803; A61B 5/02405; A61B 5/087; A61M 2021/005; A61M 21/02; A61B 5/12; A61B 3/107; A61B 5/02055

(57) ABSTRACT

Disclosed is an apparatus for therapy of cognitive impairment. In an embodiment, the apparatus includes a headwear cognition therapy unit comprising a housing having a display, a camera, a microphone, a speaker, a communication module configured to exchange data with an external service unit, an accelerometer, a three-axis gyroscope, and a memory storing data comprising firmware. The firmware is configured to use the speaker and the display to implement cognitive therapy and to detect when a patient is moving and switch the unit from VR mode to AR mode or transparent mode such that the patient can see his/her surroundings.

This application is a non-provisional of, and claims priority to, U.S. Patent Application No. 62/728,044 filed Sep. 6, 2018, the entire disclosure of which is incorporated herein by reference. The disclosures of U.S. Provisional Patent Application No. 62/728,039 filed Sep. 6, 2018 and U.S. Provisional Patent Application No. 62/728,037 filed Sep. 6, 2018 are also incorporated herein by reference in their entirety.

This application includes material which is subject to copyright protection. The copyright owner has no objection to the facsimile reproduction by anyone of the patent disclosure, as it appears in the Patent and Trademark Office files or records, but otherwise reserves all copyright rights whatsoever.

### FIELD

The present invention relates in general to the field of screening devices and methods for vision, cognitive, and auditory screening and therapy.

### BACKGROUND

Currently about 5.7 million Americans are diagnosed with Alzheimer's disease (AD), and 60 million worldwide, 11 million have some form of age-related macular degeneration (AMD) and 48 million have some degree of hearing impairment. Many citizens have simultaneous age related impairments in cognition, vision and hearing. This societal burden due to the increasing number of patients that will be diagnosed with AD alone (FIG. 1) is immense. AD is the sixth leading cause of US death, and the second leading cause of death in the United Kingdom. The Alzheimer Association points out that out of the top 10 causes of death, it's the only one without an effective treatment or cure. This apparatus fills that therapeutic void.

As illustrated in FIG. 1, the projected number of people age 65 and older in the U.S. population with Alzheimer's disease.

Few Alzheimer's disease patients are covered by government programs and 40% of family caregivers pre-decease their dependent family member.

Within the last five years, several studies have been conducted on various aspects of the behavioral systems and corresponding therapy for the patients with cognitive impairments such as early stages of Alzheimer disease and autism. For example, the US 20170319123 A1 patent application “System and methods for using mobile and wearable video capture and feedback platforms for therapy of mental disorders” presents several behavioral and mental health therapy systems that include a wearable camera and/or a variety of sensors such as accelerometer, microphone, etc. connected to a computing system having a display, audio output, holographic output and/or vibrotactile output to automatically recognize social cues from image captured by at least one camera and provide this information to the wearer via one or more outputs such as displaying an image, displaying a holographic overlay, generating an audible signal and/or generating a vibration. One of the embodiments described in said patent application comprises wearable glasses having an outward-facing camera that captures faces; other optical sensors e.g., inward-facing eye tracker to track gaze; heads-up display and audio, etc.

Yet another example is Google's Calico initiative that is aimed to tackle the spectrum of “aging-related” diseases (https://www.sunriseseniorliving.com/blog/february-2014/is-google-glass-the-future-of-alzheimers-care.aspx). Specifically, the Google Glass™ can be used as a tool to improve the lives of people who are in the early stages of Alzheimer's disease. It is suggested that the Google Glass become a “memory support system.” The device could be programmed with a sort of personal social network, and when a face is “recognized”, the individual wearing the glasses would receive cues about the other person's identity. Basic information such as name, relationship and prior interactions could help patients with Alzheimer's by offering hints about the people around them. A facial recognition system instantly connects the faces of family and friends with their names and details of their relationship. The real-time images then are relayed from an Alzheimer's Glass wearer to a remote screen so loved ones could monitor the location and safety of the user. In addition, said technology can mitigate the patients with Alzheimer's disease wandering utilizing the GPS tracking feature of Google Glass. Other augmented reality devices such as Microsoft HoloLens™, a virtual reality device such as Facebook Oculus Rift™ can be used instead of a Google Glass™ device.

The European Scientific and Medical Community are a decade or more ahead of Americans in measuring and modulating the state of stress in the human body using HRV (Heart Rate Variability). Today HRV is regarded as a global indicator of the regulatory capacity of the human. Physical, Mental and Emotional well-being can all be enhanced by harnessing HRV and consulting a cranial sacral massage therapist. There are 10,000 papers in the medical literature relating to measurement of HRV and its optimization. A commercial offshoot of the science developed at Stanford University is www.heartmath.com (Heart Math Institute) devices and software. One implementation of HRV technology to measure, analyze and bio-modulate the harmonic content of both HRV and SCP is an iPhone App found on www.iTHRVE.com. See also www.fractalfield.com and other offshoot companies. The www.iTHRVE.com biofeedback software is powerful in that it measures and harmonizes two resonant frequencies of the body. Through biofeedback, and breathing, it measures and creates phase locked resonance between the electrical frequency of the heart (manifested as blood pressure HRV) with the body's overall low frequency Mayer Wave/Cranial-Sacral Cranial Liquid Pump frequency resonance. The iPhone biofeedback software imputes the cranial sacral pump by a mathematical 3rd order “wave on wave” frequency spectrum analysis of the heart rate pulse wave to non-invasively measure the SCP wave and its signature resonant frequency. Other, less-powerful HRV technology has been developed at Stanford University and at an Austrian company named Aquaquinta. Aquaquinta has offered a suitcase-sized system to not only measure HRV, but to promote (improve) heart rate variability by scientifically synchronizing frequencies of “Light and Sound” together in free space while the patient lies on a bed wearing headphones surrounded by colored room light.

Synesthesia is a perceptual phenomenon in which stimulation of one sensory or cognitive pathway leads to automatic, involuntary experiences in a second sensory or cognitive pathway. The above-mentioned “Light and Sound” input has been used to treat synesthesia and has been used in people not affected by synesthesia to enhance cognitive performance.

In addition, Dinshah colors and syntonic phototherapy colors have been known and utilized for the therapeutic application of light.

### SUMMARY

The present invention includes a device and corresponding method that provides a multisensory and multimodal 3D experience for patients afflicted with mild to moderate cognitive impairment or dementia. The augmented reality (AR) device mitigates caregiver anxiety, and communicates with family members, professional caregivers and physicians. This autonomous device can provide an improved quality of life for all stakeholders, including the patient, the caregiver, the family and the physician. There are substantial benefits to multi-modal non-pharmacologic interventions. Such interventions include, e.g., music therapy with a music therapist, light therapy for circadian rhythm entrainment, multimodal stress reduction (color therapy, brain wave entrainment, Chakra entrainment), aromatherapy, social interaction, repetitive familiarization with people/family/pets/nature/environment/familiar objects/family albums circadian rhythm/sleep hygiene, and games, puzzles and intellectual stimulation (passive and active) as well as spiritually enhancing, culturally sensitive meditation and mantras.

In an embodiment, the presently disclosed device is capable of autonomously evaluating, at pre-determined physician prescriptive intervals, the status of the patient's hearing, vision and cognition over an extended range of his/her innate abilities. The device can provide feedback to all stakeholders (family, caregiver, physician) on the patient's ‘multisensory health status.’ In an embodiment, the invention supports emerging interventions in nutrition, environmental medicine, physical exercise, spirituality and the benefits of multifactorial non-pharmacologic interventions such as nutrition, physical exercise and stress reduction/cardiovascular disease modulation. This is important, as 99% of pharmacologic FDA drug trials for Alzheimer's disease have failed, with no new medications (beyond five existing drugs), approved over the last fourteen years.

In an embodiment, the device is configured to measure stress and induce relaxation by means of sound/light and breathing and HRV/SCP biofeedback.

In an embodiment, the device is configured to use frequencies selected from Dinshah's therapeutic twelve-sector color wheel to apply therapeutic light via the disclosed headwear cognition therapy unit. In an embodiment, the device is configured to use frequencies selected from syntonic phototherapy colors to apply therapeutic light (including near UV and near IR) via the disclosed headwear cognition therapy unit.

The presently disclosed apparatus and methods can provide invaluable information in post-market approval of new medications for the population of American dementia patients that is expected to triple by the year 2050.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a chart illustrating the projected number of people age 65 and older in the U.S. population with Alzheimer's disease.

FIG. 2 shows a perspective view illustrating a headwear cognition therapy unit.

FIG. 3 shows a block diagram illustrating configuration and operation of a headwear cognition therapy unit in accordance with an embodiment of the invention.

FIG. 4 shows a graph illustrating normalized response of a GO sensor to a modulated humid air flow.

FIG. 5 shows a flow diagram illustrating a method of testing in accordance with the present invention.

FIG. 6 shows a flow diagram illustrating a cloud platform and image and video processing in accordance with an embodiment of the invention.

FIG. 7 shows a table illustrating functions of Patient and Doctor/Technician Portals in accordance with an embodiment of the invention.

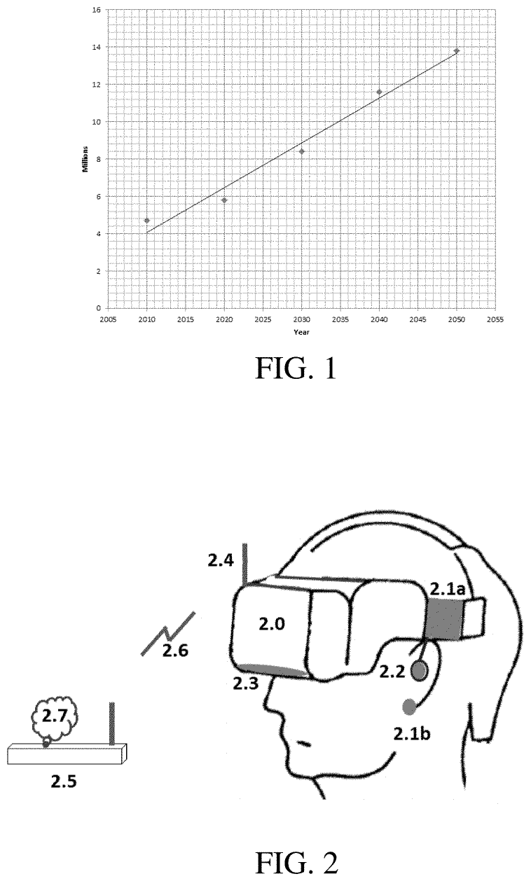

### DETAILED DESCRIPTION

Reference will now be made in detail to the preferred embodiments of the present invention, examples of which are illustrated in the accompanying drawings. The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and, such references mean at least one.

Reference in this specification to “an embodiment” or “the embodiment” means that a particular feature, structure, or characteristic described in connection with the embodiment is included in at least an embodiment of the disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

In an embodiment, the invention provides a headwear cognition therapy unit that is a multisensory multimodal AR device which serves as an inexpensive consumer device that can go to virtual reality (VR) alone for some functions if desirable (FIG. 2, 2.0).

In one embodiment said device is built on a VR platform with embedded electronics and sensors (2.1a, 2.1b, 2.2, 2.3, etc.). Examples of such VR platform include, e.g., the Oculus Rift platform available from Oculus VR of Menlo Park, Calif.

In an embodiment, the disclosed apparatus is configured to provide a closed VR environment with artificial intelligence (AI) that supports a patient's privacy and engagement and provides respite to a caregiver.

For low-vision and impaired-hearing patients, the disclosed device can be configured to provide a link to home entertainment for magnified view and sound amplification that utilizes clock feature, and photometer to gauge the needs of the cognitively impaired patient in terms of light and sound. When the device is lifted from the charging cradle, it will autonomously engage the patient announcing time, place and caregiver. An internal back facing camera checks patient's eye closure and his/her engagement via blink rate and pupil response.

Yet another embodiment contains a microphone to interact with existing technologies such as Amazon Echo® to allow patient to select music she/he wants to listen to combined with visual imagery, brain games and puzzles that are widely available for challenges at any level of cognition from AD grade 1-7 severity, and caregiver intercom.

The proposed therapy methodology includes but is not limited to music, light, anti-stress therapy and circadian rhythm/sleep hygiene treatments that are automatically driven by internal clock and timed to known benefit of establishing a circadian rhythm. For example, morning music and light activity with the high energy music and 6500 Kelvin daylight high luminance (blue) for 20 minutes can be exercised.

Treatment methodologies may include classical music, three or more different brain wave sound frequencies such as Gamma (48 Hz to 100 Hz), Beta (12 Hz to 48 Hz), Theta (8 Hz to 4 Hz) and Delta (0.5 Hz to 4 Hz) as well as a simulation of skylights and an outdoor walk in the sun that is particularly useful in confined hospital/nursing care beds without access to ambient light. In this respect, an early morning treatment may include 20 minutes of high blue light (i.e. 450-495 nm) to set the circadian rhythm via the iGCC (retinal intrinsically active ganglion cell complex), combined with stimulating music (i.e. Bach's Well Tempered Clavier) or Beta wave brain stimulation. (12 Hz to 48 Hz). This could be combined with joyful images of family, pets, nature and inspirational messages-all preprogrammed with the assistance of family members. A Mid-day treatment for stress reduction/hyperactivity may be modulated with 20-minute green dichroic (i.e. 495 nm-570 nm), or magenta light simulation calming music compositions and sounds and images of nature and animals. For the late evening sleep routine, a Delta (0.5 Hz to 4 Hz) and Theta (4 Hz to 8 Hz) frequency music, relaxing imagery and guided meditation and invocation/prayer are provided. Yet another embodiment has additional features including a built-in camera that is monitored by the system to determine if patient has fallen asleep, as well as connection to an external device to measure sleep quality or other health indicator. Yet another embodiment would have the device display 15 minutes of Baker-Miller Paint for Reducing Aggression/Agitation P-618, Schauss pink, or Drunk-Tank Pink. External devices in this regard include fitness or health monitoring/training devices, such as the Fitbit fitness trainer available from Fitbit, Inc. of San Francisco, Calif.

In an embodiment, the method described herein comprises a social interaction and repetitive familiarization with people and objects including pointing at names and faces, recognition of people and objects as well as the virtual tours of rooms of his/her house. For example, the image and sound files of spouse, sons, daughters, nieces and pets are presented to an AD patient via wireless link (FIG. 2, 2.4) from a source device (FIG. 2, 2.5). Said activity implies receiving patient's vision and sound feedback.

Yet another embodiment is enhanced with the means for measuring pre-AD changes to the eyes and vision, pupils, visual fields, saccadic eye movements and electro-physiologic testing interface interfacing with software/sensors that measure both heart rate variability (HRV) and the Mayer Wave/Sacral Cranial Pump (SCP).

In an embodiment of the invention, a device is provided for measuring HRV and SCP noninvasively using a single transducer, and resynchronizing the HRV resonance with the SCP Mayer Wave/Cranial Sacral Pump resonance. The end result is successful biofeedback and reduction of stress, thru elicitation of the relaxation response and achievement of a ‘bliss state’ heretofore achieved only by gifted cranial sacral massage therapists, who use manual digital pressure to the head and neck create a ‘healing still point’.

Proper biofeedback allows for measurement of Heart Coherence, Breath Coherence and therefore Emotional Coherence. An advantage of iThrve is that unlike Heart Math that uses breathing at a fixed 0.1 Hz breathing rate to achieve single peak coherence biofeedback, www.iTHRVE.com utilizes what they call fractal “caduceus breathing” to achieve full spectrum coherence, by measuring the relationship between traditional Heart Math single frequency HRV coherence and your full spectrum coherence (measuring the cranial sacral pump or SCP). physiologic state. Within 4 to 8 minutes, the end result is reduction of stress, thru elicitation of the relaxation response heretofore achieved only by gifted cranial sacral massage therapists.

The presently disclosed head-mounted display therefore can utilize a built-in blood pressure pulse transducer (HRV) that could be an ear clip transducer or a transducer built into the head strap or mounted on the forehead, that does not require extra effort for attachment. In addition, the disclosed head-mounted display can use Bluetooth wireless connectivity for other transducer inputs such as a Polar H7 chest-strap pulse transducer (cross chest 2 lead EKG), i.e. finger plethysmograph (the best transducer for SCP measurement). A key health goal is expanding Heart Rate Variability (HRV) using biofeedback, breath, color, sound, sacred geometry images and mantras etc.

A Bluetooth finger pulse oximeter can be used to measure both HRV and the Mayer Wave but it requires a steady hand with no change in room lighting. Alternatively, an iWatch or other smart watch may be used to provide this capability.

One headwear cognition therapy unit embodiment has pulse and breathing sensors that provide bio-feedback with the aim of real time therapeutically routine correction or cancellation if needed. These biometric sensors measure temperature, ear lobe oxygen saturation and pulse, as well as breathing frequency (i.e. directional microphone) to autonomously provide the patient with a multisensory, multimodal therapeutic experience.

In an embodiment, the headwear cognition therapy unit is capable of pulsed photo-bio-modulated infrared light with the wave length of 660 nm/780 nm to heal the retina. In an embodiment, the headwear cognition therapy unit is capable of pineal gland reactivation using specific sound and light frequencies to control the level of melatonin.

In an embodiment, the headwear cognition therapy unit is capable of interfacing with the Max Luscher Institute 8 color test (circa 1945), a test that reveals personality through color. The late Max Luscher (1923-2017) was a swiss psychotherapist who demonstrated that color preference is not subjective but objective. This technology measures a person's psychophysical state, his or her ability to withstand stress, to perform, and to communicate. It uncovers the cause of psychological stress, which can lead to physical symptoms.

The headwear cognition therapy unit and method described herein can be part of a system that is able to track use of the headwear cognition therapy unit using an internet cloud, track progress with embedded brain game software, communicate with a medical team, note declines or improvements in patient's cognitive abilities, perform diagnostics using the corresponding software and machine learning algorithms, and create a larger community of concerned family members and distant relatives. Another embodiment contains a neck strap and GPS location beacon.

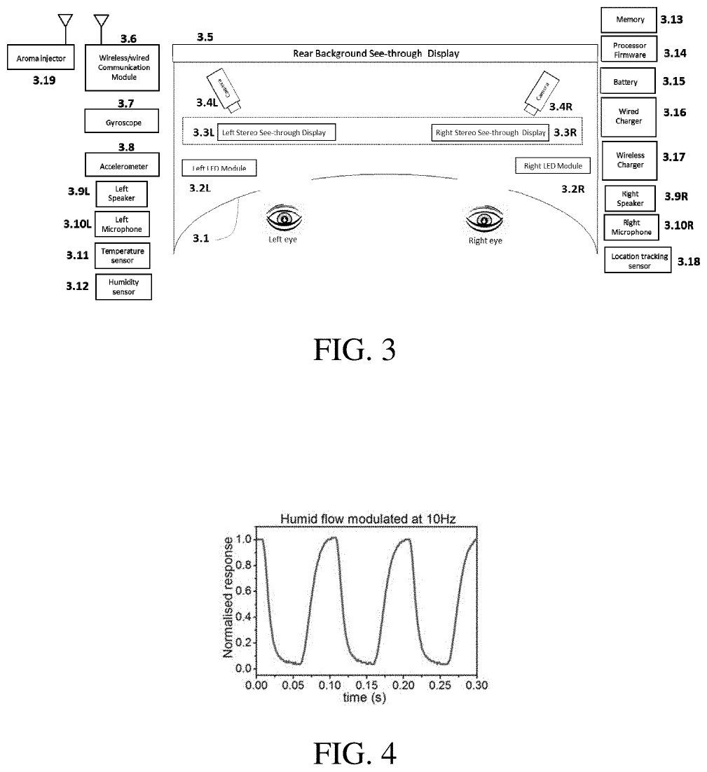

A detailed representation of a headwear cognition therapy unit is shown in FIG. 3. In FIG. 3, the unit includes a housing 3.1, LED modules 3.2L and 3.2R, and stereo displays 3.3L and 3.3R (or non-stereo displays or a single display to cover both eyes). A processor and the corresponding firmware are shown at 3.14. A built-in memory is shown at 3.13. The eye monitoring cameras 3.4L and 3.4R feature optional autofocus functionality to obtain a perfect image of the eye, with a high enough frame rate to detect and measure blinking. Alternatively, a single camera in the middle can cover both eyes. Close-to-the-ear stereo speakers 3.9L and 3.9R can be ear mounted, next to the ear, directional speakers directed away from the ear, or use bone conduction. Stereo microphones 3.10L and 3.10R are provided. A wireless and wired communication module 3.6 communicates with other devices including but not limited to a central “service unit” (FIG. 2, 2.5). A rechargeable battery 3.15 is provided. The wired and wireless charging modules are shown at 3.15 and 3.16 respectively. The accelerometer is shown at 3.8 and gyroscope at 3.7. Temperature, pulse and oxygen sensors 3.11 are provided. A location tracking sensor is shown at 3.18, a humidity sensor is shown at 3.12, and an aromatherapy module comprising a wirelessly controlled aroma injector with scent capsule(s) is shown at 3.19.

Alternatively, the aromatherapy module can be part of the source device (FIG. 2, 2.7) being wirelessly activated using Bluetooth, Wi-Fi or other wireless technology from headwear cognition therapy unit (FIG. 2, 2.6). In some embodiment the activation message is correlated with the images or video that is shown to the patient.

The eye monitoring cameras are intended for photographing still images and video capturing. The ear mounted stereo speakers and stereo microphones are used for the hearing screening as well as for audio communication between the system and a patient.

In some embodiments, a processor controls the routine sequences, displaying images and audio signals, performs control functions during the headwear cognition therapy unit communication, as well as the information post processing.

In some embodiments, said accelerometer is used to capture the patient input provided by patient's head movement. Further, a three-axis gyroscope is used for patient's head position identification and monitoring—to alert caregiver if patient arises from their chair or bed.

In some embodiment, when motion is detected the unit will automatically switch to the AR/transparent mode, so the patient can see his/her surrounding and optionally alert the care giver.

Yet another headwear cognition therapy unit embodiment comprises the breathing sensor (FIG. 2, 2.3) that is based on the ultrafast graphene oxide (GO) sensor measuring humidity while patient breathes and/or speaks. Normalized response of a 15 nm thick GO sensor to a modulated humid air flow at 10 Hz is shown in FIG. 4.

For heart beat rate, the ear lobe sensor (FIG. 2, 2.1b) can be used but it is more convenient to employ a built-in sensor in the headwear cognition therapy unit that provides measurement at the temple area (FIG. 2, 2.1a). Further, the temperature can be measured at the same area.

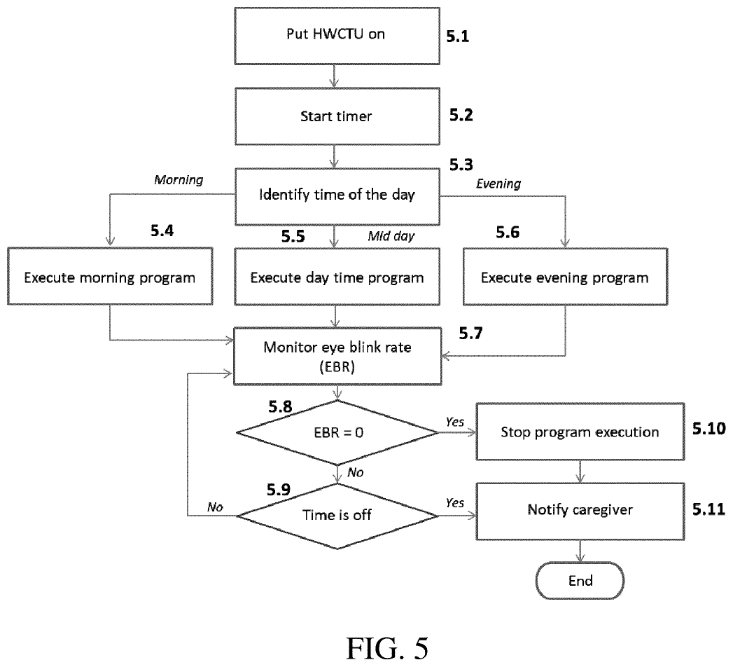

The method flowchart is depicted in the FIG. 5.

In an embodiment, the headwear cognition therapy unit is configured to apply therapy via frequencies selected from Dinshah's therapeutic color wheel to apply therapeutic light. For example, the headwear cognition therapy unit can be configured to apply light in the blue/violet range of the spectrum from 380 nm to 425 nm for anti-inflammatory treatment to treat eyelid lesions and blepharitis, blue light to treat traumatic brain injuries migraine headache, and burns. The headwear cognition therapy unit can be configured to apply light in the red range of the spectrum from 625 nm to 740 nm for ocular photo-biomodulation treatment for increased blood flow through, e.g., kidneys, thyroid, and the liver (e.g., for non-alcoholic fatty liver disease). Light in the red range has been used to increase mitochondria function. The headwear cognition therapy unit can be configured to apply light in the green range of the spectrum from 520 nm to 565 nm for antiseptic treatment. The headwear cognition therapy unit can be configured to apply light in the purple range of the spectrum (red light from 625 nm to 740 nm plus blue light from 445 nm to 520 nm) for treatment of high blood pressure and pain. The headwear cognition therapy unit can be configured to apply light in the indigo range of the spectrum from 425 nm to 445 nm for treatment of hemorrhage and pain. The headwear cognition therapy unit can be configured to apply light in the magenta range of the spectrum for equilibrating and calming treatment to treat pain.

The headwear cognition therapy unit can be configured to apply light in the turquoise range of the spectrum for, e.g., healing and treatment of optic nerve trauma and traumatic brain injury. In this respect, 485 nm light (Dinshah ultra green) can be applied to activate the intrinsically active ganglion cell complex (iPGC).

In an embodiment, the device is configured to use frequencies selected from early 20th century syntonic phototherapy colors to apply therapeutic light via the disclosed headwear cognition therapy unit. In an embodiment, the invention provides a head mounted autonomous device with enhanced timing, sensors, and data management capability, the head-mounted autonomous device being configured to therapeutically apply the prescribed short duration of both synesthetic light and sound. The device can be configured to use such light, sound and vibration therapy together e.g., synesthesia.

In an embodiment, the disclosed headwear cognition therapy unit is configured to apply sound/musical compositions (i.e. Mozart, Verdi) at a frequency of 432 Hz that induces the theta/alpha wave state of the brain EEG. This sound frequency resonates with the planetary Schumann Resonance at a frequency of 7.8 Hz to 8 Hz depending on latitude. The 432 Hz sound frequency has been known to reduce anxiety, lower blood pressure and heart rate and activate DNA/healing. It was used in the instruments of the ancient Egyptians, Tibetan monks (bowls), Greeks and in Stradivarius violins. This frequency has also been known to encourage synchronicity between the hemispheres of the brain and increase creativity, insight and intuition.

In an embodiment, the disclosed headwear cognition therapy unit is configured to apply both sound or musical compositions at an isochromic frequency of 432 Hz in combination with light therapy. In this embodiment, the disclosed headwear cognition therapy unit is configured to apply light at a frequency of 620 nm (ocre or orange). Ocre is the color of happiness, sociability and youth. The 620 nm light wavelength has been shown to increase UV light absorption and provide other beneficial health effects. In an embodiment, the disclosed headwear cognition therapy unit is configured to apply sound/musical compositions at a frequency of 432 Hz in combination with light at wavelength 620 nm. In a further embodiment, the subject views a three-dimensional sacred geometric ocre colored 40 Hz Flickering/Rotating Phi Stellated Dodecahedron (Twelve Pentagon (five-sided face)) ‘platonic solid’ figure rotating in space.

In another embodiment, the disclosed headwear cognition therapy unit is configured to apply sound at a frequency of 531 Hz in combination with light therapy. 531 Hz is one of the ancient Solfeggio musical frequencies called the “love frequency” or “miracle tone” & “DNA repair frequency” as the sound 528 Hz increases UV light absorption in DNA. The frequencies for sound therapy discussed above can be combined with the corresponding light wavelength of green (530 nm) and implemented in the headwear cognition therapy unit via a speaker connected to a source that plays classical music in a corresponding key. For example, 528 Hz (531.5 Hz) is the isochronic tone C and corresponds to the key of C or middle heart chakra (i.e. Fantasy in C by Robert Schumann) In an enhanced synesthesia embodiment, the subject would view a rotating 3D 40 Hz green (530 nm wavelength) icosahedron.

In another embodiment, the disclosed headwear cognition therapy unit is configured to perform testing by applying UVA light at 280 nm-310 nm to the cornea, take measurements or photographs of the cornea, and relate the measurements or image to the amount of calcification in the heart. In this respect, the system can be configured to calculate an Agatston score or other Coronary Artery Calcium (CAC) score based on the results of such testing.

In an embodiment, the present invention provides an apparatus for therapy of cognitive impairment, the apparatus having a housing that includes a full set or a subset of the following components: a display, a left and a right inward pointing camera or a single camera, a left and a right LED module, a stereo microphone, a stereo speaker, a wired communication module, a wireless communication module, an accelerometer, a three-axis gyroscope, a processor, firmware, a memory, a temperature sensor, a humidity sensor, a location tracking sensor, a power supply, a rechargeable battery, a wired charging component, a wireless charging component, a service unit supporting wireless exchange of information with the headwear cognition therapy unit, and an aromatherapy module having a wirelessly controlled aroma injector with the scent capsule(s). The humidity sensor may be used for patient rate of breath. Information exchanged with the headwear cognition therapy unit may include image, video and music files. The display may include left and a right stereo displays, non-stereo displays, or a single display.

In an embodiment, the invention comprises a method to detect when a patient is moving and switch the unit from VR to AR or transparent mode, so that the patient can see his/her surroundings. In an embodiment, a method for therapy of cognitive impairment includes the steps of putting a headwear cognitive therapy unit on the patient's head, establishing connection with the service unit, starting a timer for a predefined time period, identifying the time of the day, compiling a therapy routine as a function of the time of the day, executing the therapy routine, monitoring eye blink rate, monitoring timer expiration signal, and stopping routine execution and notifying a caregiver when one of the conditions is TRUE: eye blink rate is equal to zero or a timer expiration signal is received.

In an embodiment, the invention described above is configured for prescriptive autonomous therapy wherein a doctor may work with a consumer, with tracking. These embodiments may be used to implement medical or non-medical protocols for stress reduction, depression, PTSD, Traumatic Brain Injury (TBI), and minimizing photosensitive migraines and epileptic seizures as well as autism behavior modulation. The system may be configured to facilitate conscious breathing with biofeedback. The system may be configured to measure HRV (heart rate variability and cranial sacral still point. It may also be configured to support meditation induction for mindfulness, Eye Movement Desensitization Reprocessing (EMDR), resonating to the sacred geometry of art and architecture, and resonating to images and sounds of nature and animals.

The prescriptive autonomous therapy embodiments may further be configured to implement medical or non-medical protocols to combat Neurologic Disorders. In this respect, these embodiments may be combined with stress and depression reduction mode described above. and can be configured to implement medical or non-medical protocols to combat Alzheimer's or Parkinson's disease, or can be configured for nootropic therapy. The system may be configured for gamma wave entrainment, or combined 40 Hz Light/Near IR/Sound/Cranial Vibration.

The prescriptive autonomous therapy embodiments may further be configured to implement medical or non-medical protocols to combat ophthalmic disorders using photobiomodulation. Such embodiments may also be combined with stress and depression reduction mode described above. Ophthalmic disorders addressable using such embodiments include age-related macular degeneration, glaucoma (using optic nerve stimulation), dry eye/Meibomitis (using red/IR stimulation of eyelids).

The prescriptive autonomous therapy embodiments may further be configured to implement medical or non-medical protocols for anti-aging. Such embodiments may also be combined with stress and depression reduction mode described above. Anti-aging protocols addressed by these embodiments include those that slow biological aging, such as telomere length extension protocols, protocols that improve systemic cell mediated immunity, and those that lower inflammation.

In an embodiment, the invention described above is configured for artificial intelligence eye/cognitive screening and research In particular, the system described above may be used alongside a retinal fundus camera for artificial intelligence telehealth eye and neuro screening, in applications such as autonomous diabetic retinopathy screening testing centers with cloud-based AI analysis. In another embodiment, the device is configured for efficient evaluation of pharmaceuticals (or nutrients) without the necessity of constructing/maintaining a testing center.

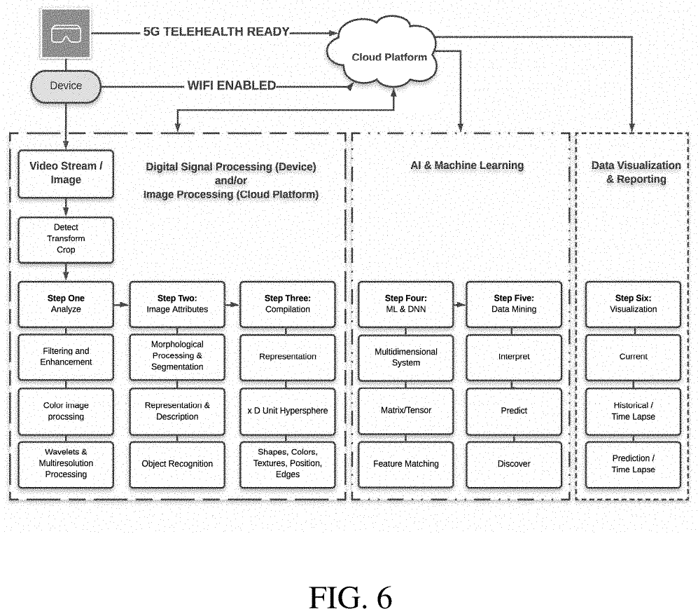

As noted above, the headwear cognition therapy unit and method described herein can be part of a system that is able to track use of the headwear cognition therapy unit using an internet cloud, track progress with embedded brain game software, communicate with a medical team, note declines or improvements in patient's cognitive abilities, perform diagnostics using the corresponding software and machine learning algorithms, and create a larger community of concerned family members and distant relatives. FIG. 6 shows a flow diagram illustrating a cloud platform and image and video processing in accordance with an embodiment of the invention. In an embodiment, the device includes onboard DSP (Digital Signal Processors) for real time video processing and conversion of video to processable data. In such embodiments, the device provides not just ability to save a raw video but actual live data points in the video that can be stored in databases and reviewed. Video and image processing can be performed on the cloud side for accuracy. Each test will go through the diagramed process shown in FIG. 6. The video and images are collected and processed from the cameras on the headwear cognition therapy unit at the time of screening. Each AI/ML (Machine Learning) attempt gathers data, adds data, and learns from all previous screening for each test.

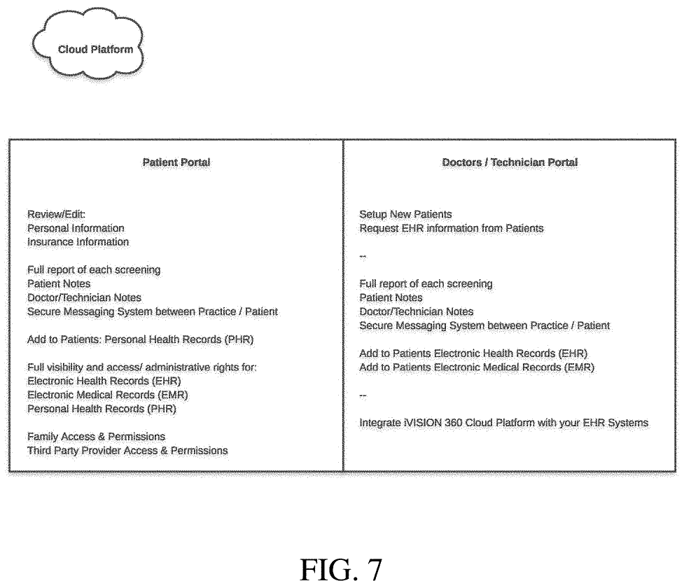

FIG. 7 shows a table illustrating functions of Patient and Doctor/Technician Portals in accordance with an embodiment of the invention.

The present invention is described above with reference to block diagrams and operational illustrations of methods and devices for autonomous multisensory testing. It is understood that each block of the block diagrams or operational illustrations, and combinations of blocks in the block diagrams or operational illustrations, may be implemented by means of analog or digital hardware and computer program instructions. These computer program instructions may be stored on computer-readable media and provided to a processor of a general-purpose computer, special purpose computer, ASIC, or other programmable data processing apparatus, such that the instructions, which execute via the processor of the computer or other programmable data processing apparatus, implements the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

At least some aspects disclosed can be embodied, at least in part, in software. That is, the techniques may be carried out in a special-purpose or general-purpose computer system or other data processing system in response to its processor, such as a microprocessor, executing sequences of instructions contained in a memory, such as ROM, volatile RAM, non-volatile memory, cache or a remote storage device. Functions expressed in the claims may be performed by a processor in combination with memory storing code and should not be interpreted as means-plus-function limitations.

Routines executed to implement the embodiments may be implemented as part of an operating system, firmware, ROM, middleware, service delivery platform, SDK (Software Development Kit) component, web services, or other specific application, component, program, object, module or sequence of instructions referred to as “computer programs.” Invocation interfaces to these routines can be exposed to a software development community as an API (Application Programming Interface). The computer programs typically comprise one or more instructions set at various times in various memory and storage devices in a computer, and that, when read and executed by one or more processors in a computer, cause the computer to perform operations necessary to execute elements involving the various aspects.

A machine-readable medium can be used to store software and data which when executed by a data processing system causes the system to perform various methods. The executable software and data may be stored in various places including for example ROM, volatile RAM, non-volatile memory and/or cache. Portions of this software and/or data may be stored in any one of these storage devices. Further, the data and instructions can be obtained from centralized servers or peer-to-peer networks. Different portions of the data and instructions can be obtained from different centralized servers and/or peer-to-peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine-readable medium in entirety at a particular instance of time.

Examples of computer-readable media include but are not limited to recordable and non-recordable type media such as volatile and non-volatile memory devices, read only memory (ROM), random access memory (RAM), flash memory devices, removable disks, magnetic disk storage media, optical storage media (e.g., Compact Disk Read-Only Memory (CD ROMS), Digital Versatile Disks (DVDs), etc.), among others.

In general, a machine-readable medium includes any mechanism that provides (e.g., stores) information in a form accessible by a machine (e.g., a computer, network device, personal digital assistant, manufacturing tool, any device with a set of one or more processors, etc.).

In various embodiments, hardwired circuitry may be used in combination with software instructions to implement the techniques. Thus, the techniques are neither limited to any specific combination of hardware circuitry and software nor to any particular source for the instructions executed by the data processing system.

As used herein, and especially within the claims, ordinal terms such as first and second are not intended, in and of themselves, to imply sequence, time or uniqueness, but rather are used to distinguish one claimed construct from another. In some uses where the context dictates, these terms may imply that the first and second are unique. For example, where an event occurs at a first time, and another event occurs at a second time, there is no intended implication that the first time occurs before the second time. However, where the further limitation that the second time is after the first time is presented in the claim, the context would require reading the first time and the second time to be unique times. Similarly, where the context so dictates or permits, ordinal terms are intended to be broadly construed so that the two identified claim constructs can be of the same characteristic or of different characteristic.

While some embodiments can be implemented in fully functioning computers and computer systems, various embodiments are capable of being distributed as a computing product in a variety of forms and are capable of being applied regardless of the particular type of machine or computer-readable media used to actually effect the distribution.

The above embodiments and preferences are illustrative of the present invention. It is neither necessary, nor intended for this patent to outline or define every possible combination or embodiment. The inventors have disclosed sufficient information to permit one skilled in the art to practice at least one embodiment of the invention. The above description and drawings are merely illustrative of the present invention and that changes in components, structure and procedure are possible without departing from the scope of the present invention as defined in the following claims. For example, elements and/or steps described above and/or in the following claims in a particular order may be practiced in a different order without departing from the invention. Thus, while the invention has been particularly shown and described with reference to embodiments thereof, it will be understood by those skilled in the art that various changes in form and details may be made therein without departing from the spirit and scope of the invention.

What is claimed is:

1. An apparatus for therapy of cognitive impairment, the apparatus comprising:
a headwear cognition therapy unit comprising a housing having:
i) a display;
ii) a camera;
iii) a microphone;
iv) at least one speaker;
v) at least one communication module configured to exchange data with an external service unit;
vi) an accelerometer;
vii) a three-axis gyroscope;
viii) a memory storing data comprising firmware configured to use the speaker and the display to implement cognitive therapy and to detect when a patient is moving and switch the unit from VR mode to AR mode or transparent mode such that the patient can see his/her surroundings;
ix) a processor executing said firmware;
x) a location tracking sensor; and,
xi) a power supply component having a rechargeable battery.

2. The apparatus for therapy of cognitive impairment according to claim 1, wherein said firmware comprises code configured to utilize artificial intelligence to support a patient's privacy and engagement.

3. The apparatus for therapy of cognitive impairment according to claim 1, wherein said firmware comprises code configured to autonomously evaluate, at pre-determined physician prescriptive intervals, the status of the patient's hearing, vision and cognition over an extended range of their innate abilities.

4. The apparatus for therapy of cognitive impairment according to claim 1, further comprising an aromatherapy module comprising a wirelessly controlled aroma injector operatively connected to one or more scent capsules.

5. The apparatus for therapy of cognitive impairment according to claim 1, wherein said firmware comprises code configured to measure stress and induce relaxation by means of sound, light or breathing biofeedback.

6. The apparatus for therapy of cognitive impairment according to claim 1, wherein the apparatus is configured to use frequencies selected from Dinshah's therapeutic color wheel to apply therapeutic light via the headwear cognition therapy unit.

7. The apparatus for therapy of cognitive impairment according to claim 1, wherein the apparatus is configured to use frequencies selected from syntonic phototherapy colors to apply therapeutic light via the disclosed headwear cognition therapy unit.

8. The apparatus for therapy of cognitive impairment according to claim 1, further comprising an electro-physiologic testing interface interfacing with software or sensors that measure both heart rate variability and Mayer Wave/Sacral Cranial Pump.

9. The apparatus for therapy of cognitive impairment according to claim 8, wherein the device is configured to measure heart rate variability and Mayer Wave/Sacral Cranial Pump noninvasively using a single transducer.

10. The apparatus for therapy of cognitive impairment according to claim 9, wherein the device is configured to resynchronize heart rate variability and Mayer Wave/Sacral Cranial Pump resonance.

11. The apparatus for therapy of cognitive impairment according to claim 1, further comprising a blood pressure pulse transducer.

12. The apparatus for therapy of cognitive impairment according to claim 1, further comprising a humidity sensor configured to measure patient rate of breath.

13. The apparatus for therapy of cognitive impairment according to claim 1, further comprising a light source configured to deliver photo-bio-modulated infrared light with a wave length of 660 nm/780 nm to a retina of a person wearing the headwear cognition therapy unit.

14. The apparatus for therapy of cognitive impairment according to claim 1, where information exchange with the headwear cognition therapy unit comprises exchange of image files, video files, or music files.

15. The apparatus for therapy of cognitive impairment according to claim 1, wherein the display comprises left and a right stereo displays, non-stereo displays, or a single display.

16. The apparatus for therapy of cognitive impairment according to claim 1, wherein the camera comprises left and a right inward-pointing cameras.

17. The apparatus for therapy of cognitive impairment according to claim 1, further comprising a left and a right LED module.

18. The apparatus for therapy of cognitive impairment according to claim 1, wherein said speaker comprises first and second speakers operating in stereo.

19. The apparatus for therapy of cognitive impairment according to claim 1, wherein said at least one communication module comprises a wired communication module and a wireless communication module.

20. The apparatus for therapy of cognitive impairment according to claim 1, wherein said housing further contains a temperature sensor.

21. The apparatus for therapy of cognitive impairment according to claim 1, wherein said housing further contains a humidity sensor;

22. The apparatus for therapy of cognitive impairment according to claim 1, wherein said power supply component further comprises a wired charging component and a wireless charging component.

23. The apparatus for therapy of cognitive impairment according to claim 1, wherein said apparatus is configured to perform testing by applying UVA light at 280 nm-310 nm to a cornea of a patient, take measurements or image of the cornea, and relate the measurements or image to the amount of calcification in the heart.

24. The apparatus for therapy of cognitive impairment according to claim 23, wherein the apparatus is configured to calculate an Agatston score or other coronary artery calcium score based on results of said testing.

25. A method for therapy of cognitive impairment, the method comprising the steps of:
placing a headwear cognitive therapy unit on a patient's head;
establishing connection from said headwear cognitive therapy unit to a service unit;
starting timer for predefined time period;
identifying a time of the day;
compiling a therapy routine as a function of said time of the day;
executing said therapy routine;
monitoring eye blink rate;
monitoring a timer expiration signal;
stopping routine execution and notifying a caregiver when one of the following conditions is TRUE: eye blink rate is equal to zero or timer expiration signal is received.
